(19) United States
(12) Patent Application Publication
(10) Pub. No.: US 2025/0017531 A1
(43) Pub. Date: Jan. 16, 2025

(54) Smart rings and construction methods therefor

(71) Applicant: Senbiosys, Neuchâtel (CH)

(72) Inventors: Anthony Barison, Lausanne (CH); Antonino Caizzone, Milvignes (CH); Assim Boukhayma, Neuchâtel (CH)

(21) Appl. No.: 18/769,698

(22) Filed: Jul. 11, 2024

Related U.S. Application Data
(60) Provisional application No. 63/513,123, filed on Jul. 12, 2023.

(51) Int. Cl. A61B 5/00

(52) U.S. Cl. CPC A61B 5/6826; A61B 2560/0209; A61B 2560/0462; A61B 2562/0219; A61B 2562/04; A61B 2562/12; A61B 2562/166; A61B 2562/227

(57) ABSTRACT

A ring for photoplethysmographic (PPG) sensing performs transmissive PPG and/or reflective PPG. In particular, a ring and assembly methods therefor are described including for example connecting a rim to an inner ring via a bonding process, laying a printed circuit board (PCB) on the inner ring, aligning optical sensors of the PCB with ports of the inner ring, providing electrical connections between contact points on the PCB and the rim, and inserting an assembly including the ring, inner ring and PCB into an outer shell.

### RELATED APPLICATIONS

This application claims the benefit under 35 USC 119 (e) of U.S. Provisional Application No. 63/513,123, filed on Jul. 12, 2023, which is incorporated herein by reference in its entirety.

### BACKGROUND OF THE INVENTION

Nowadays, wearable devices, such as fitness trackers or smartwatches, with optical heart rate sensors, are becoming common. Even rings are now available with these sensors.

The technology behind these sensors is called photoplethysmography (PPG), which is an optical measurement technique used to detect blood volume changes in living tissues. A PPG sensor requires a few optoelectronics components, such as a light source, e.g. light-emitting-diode (LED), to illuminate the living tissue, a photodetector (PD) to track any light intensity variation due to the blood volume change, and an analog front-end (AFE) for signal conditioning and processing. Today, the importance of PPG for medical monitoring is proven by the number of primary vital signs directly or indirectly that can be resolved by it.

In a typical example, the PPG signal is obtained by shining light from the LED at a given wavelength, in the visible or near-infrared range, into the finger, wrist, forehead, or ear lobes. The PPG sensor's photodetector detects the light transmitted through (transmissive PPG) or reflected (reflective PPG) from the tissue and transforms it into a photogenerated current. The detected signal, i.e. PPG signal, has two different components: a large DC (quasi-static) component corresponding to the light diffusion through tissues and non-pulsatile blood layers, and a small AC (pulsatile) part due to the diffusion through the arterial blood. The AC component is only a very small fraction (typically 0.2% to 2%) of the DC one, meaning the AC component is 500 to 50 times smaller than the DC component. This AC/DC ratio mostly depends on the sensor's location on the body, and the LED wavelength, and weakly on the skin tone. This AC/DC ratio is often called perfusion-index (PI) and ultimately sets one of the challenges for any PPG readout system. Indeed, the AC component carries most of the biomedical information. Low PI values lead to reduced signal fidelity, complicated signal processing schemes and larger power consumption.

U.S. Pat. Pub. No. US 2023/0008487, entitled Sensing System and Method for Smart Rings Employing Sensor Spatial Diversity, by the inventors hereof, describes devices and operating methods allowing a better photoplethysmographic sensing on the finger or other digit or other location where transmissive PPG and/or reflective PPG is possible. These described devices can enable lower power consumption, higher fidelity, and/or greater versatility to different use cases and users' specificities. This PPG system takes advantage of sensor spatial diversity to enhance the quality and the reliability of the PPG measurements in smart rings, for example.

### SUMMARY OF THE INVENTION

As mentioned, the (sensing) body location of the PPG sensor affects the PI and the signal quality. Different measurement sites can be used including the wrist, the finger and the ear region (both the lobe and the canal). The finger, however, has been shown to be among the best locations in terms of PI, at a given optical power, providing better biomedical sensing.

In addition to the intrinsically larger PI, the finger is the standard location when it comes to oxygen saturation monitoring in medical environments, which is based on finger clips using the transmissive PPG method.

If on one hand, wearable devices usually embed reflective PPG thanks to its intrinsic lower power consumption and easiness to place on the body, on the other hand the transmissive method remains the standard for accurate oxygen saturation analysis. Indeed, reflective PPG can be affected by many factors, including reduced blood perfusion due to hypothermia (cold fingers), applied pressure and motion artefacts (MA). MA can be of various types and ultimately distort the PPG signal setting a challenge for PPG systems. MA can be periodic or non-periodic and can present a much larger amplitude than the AC component of the PPG signal. In addition, MA can fall within the same frequency band as the HR. For these reasons, MA need to be minimized and/or compensated as much as possible.

The present approach relates to designs for smart rings that often integrate multiple PPG sensors around the ring's perimeter and in particular strategies for assembly and manufacture of those rings.

In general, according to one aspect, the invention features a ring system comprising a printed circuit board (PCB), an inner ring holding the printed circuit board, and an outer shell mounted over the printed circuit board and the inner ring.

In specific embodiments, ports are provided in the inner ring to provide optical paths for optical sensors of the printed circuit board. Additionally, the inner ring might have a U shape cross-section. A rim is provided that is assembled onto the inner ring via bonding.

In one example, the outer shell is metal and is preferably electrically connected to the printed circuit board to function as an antenna for the Bluetooth-low-energy communications block BLE.

In general, according to one aspect, the invention features an assembly process for a ring, comprising laying a PCB on an inner ring, aligning optical sensors of the PCB with ports of the inner ring, and inserting an assembly including the inner ring and PCB into an outer shell.

In examples, the process also may include connecting a rim to the inner ring via a bonding process. Electrical connections could also be provided between the PCB and the outer shell to function as an antenna.

Clamps can be employed to retain the PCB in the inner ring. The outer shell and the rim can be electrically connected with conductive glue. In addition, the outer shell can be provided in two pieces.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a side cross-sectional schematic view perpendicular to the axis of a sensor ring system, showing one example of a smart ring;

FIG. 2 is a side cross-sectional schematic view perpendicular to the axis of a sensor ring system, showing another example of a smart ring;

FIG. 3 is a schematic plan view of a PPG sensor 200 for the smart ring;

FIGS. 4A, 4B, 4C, and 4D are assembly drawings showing one design for a compact smart ring with FIG. 4A being an exploded view, FIG. 4B being a side cross-sectional view perpendicular to the axis of the ring, FIG. 4C being a cross-sectional view at B-B in FIG. 4B and FIG. 4D being a cross-sectional view at C-C in FIG. 4B;

FIG. 5 is an assembly drawing showing an exploded view of another design for a compact smart ring;

FIG. 6 is an assembly drawing showing an exploded view of another design for a compact smart ring;

FIGS. 7A, 7B, 7C, and 7D are assembly drawings showing one design for a compact smart ring with FIG. 7A being an exploded view, FIG. 7B being a side cross-sectional view perpendicular to the axis of the ring, FIG. 7C being a cross-sectional view at B-B in FIG. 7B and FIG. 7D being a cross-sectional view at C-C in FIG. 7B;

FIGS. 8A, 8B, 8C, and 8D show four potential cross sections of different embodiments of the ring;

FIGS. 9A, 9B, and 9C show four potential cross sections of different embodiments of the ring; and

FIG. 10 is an exploded view showing another design for a compact smart ring.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

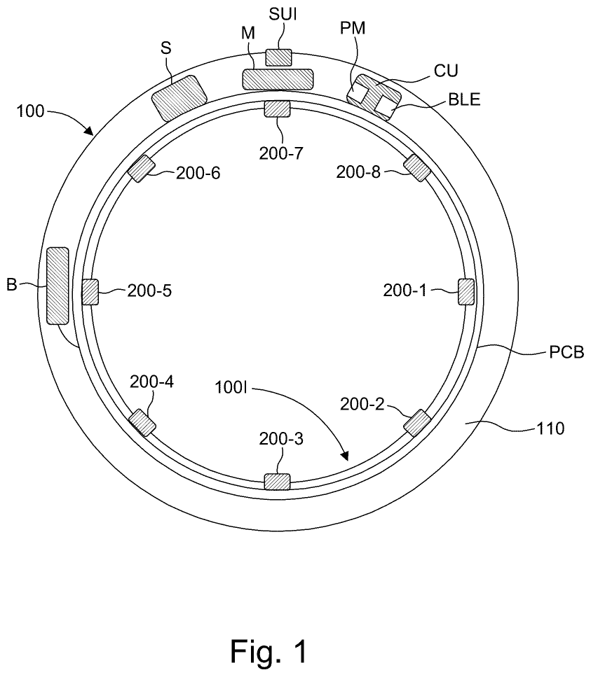

FIG. 1 shows an example of the sensor ring system 100.

In general, the sensor ring includes a ring or annual-shaped ring body 110 having an inner bore 110I sized to receive a human digit such as a finger, and specifically an index finger, middle finger, ring finger, or little finger or pinkie. The ring body 110 is typically a molded plastic or ceramic material, but could also be metal.

An array of PPG sensor units 200-1 to 200-8 are distributed around the inner bore and directed inwardly in order to perform PPG sensing of the digit inserted into the inner bore 110I. In the illustrated example, the sensor units 200-1 to 200-8 are evenly arrayed in a ring concentric to the ring body 110 and mounted to a flexible printed circuit board PCB that is affixed to an inner side of the in the inner bore 110I.

A control unit CU is further installed on the PCB. The control unit CU controls the triggering of and readout from the PPG sensors 200. In one mode of operation, the control unit CU alternates between a transmissive PPG mode and reflective PPG mode. In one example, the control unit controls the sensor units 200 to perform a combination of transmissive and reflective PPG sensing as described in US-2023-0008487 A1, which is incorporated herein by this reference in its entirety.

The control unit CU preferably includes a Bluetooth-low-energy communications block BLE and a power management block PM, for communicating data with the external world and regulating system power, respectively.

The control unit CU stores sensor readings to a memory unit M also on the PCB. The memory unit also contains the operating instructions for the system set-up and other firmware components required for operation.

A battery B provides power to the system via the PCB, and is generally controlled by the power management unit PM in order to provide for extended operation.

A sensor block S has inertial sensors, including a three axis accelerometer and a three axis gyroscope. In some cases, the accelerometer and the gyroscope each have more than three axes, possibly six axes. In addition, the sensor block S also preferably has a user temperature sensor and an ambient temperature sensor, for separate body and environmental temperature detection.

A smart user interface block SUI provides active feedback from the device to the user and also receives user control input. In some examples, the SUI block includes a visible LED array and a touch sensor system, such as a capacitive touch sensor array.

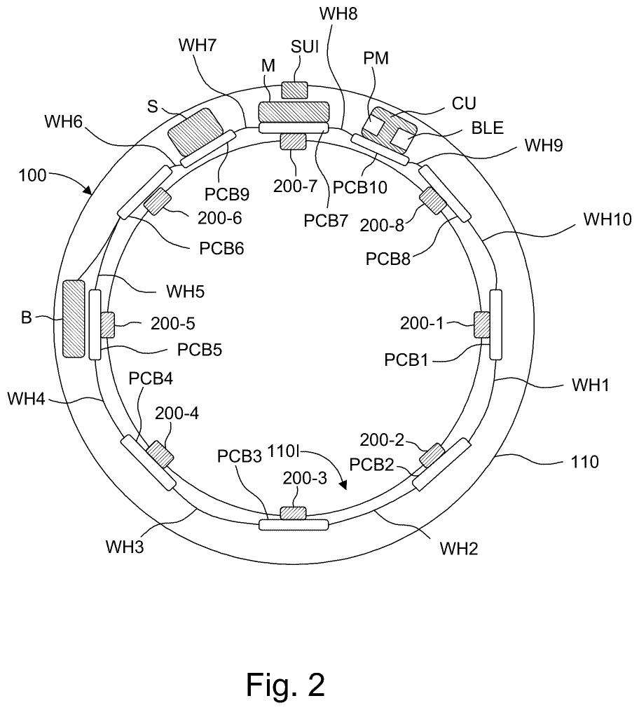

FIG. 2 shows another example of the sensor ring system 100. This uses a series of small independent PCBs, i.e., PCB1-PCB10 for each of the sensor units 200, the battery B, the memory M and smart user interface block SUI, the control unit CU, and sensor block S. Each of these PCBs are interconnected with wiring harnesses WH1-WH10 in a daisy-chained fashion.

In still other examples, the independent PCBs are replaced with one or more flexible PCBs. In both cases, flexible PCBs can be used to allow a better integration into the system.

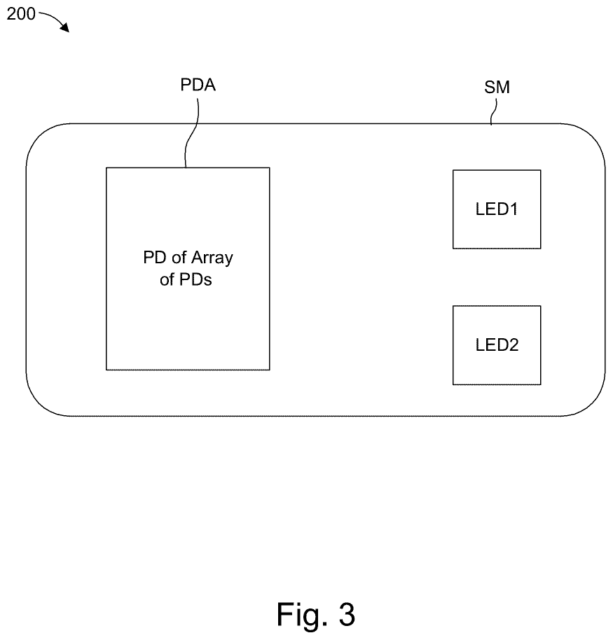

FIG. 3 shows an exemplary configuration for each of the PPG sensors 200.

In a typical implementation, each PPG sensor 200 includes at least one and preferably multiple photodiodes. In one example, an array of at least 8 by 8 photodiodes PDA is provided on a submount SM.

Each sensor 200 also includes one or more light emitters LED1, LED2 such as light emitting diodes (LED) or vertical cavity surface emitting lasers (VCSELs) installed on the submount SM, next to a light sensor such as a photodiode or photodiode array PDA, also on the submount. Moreover, the light emitters LED1, LED2 can operate at different wavelengths. In the illustrated example, two light emitters LED1 and LED2 are provided on the submount SM that emit at different wavelength in the visible and/or infrared.

In a current example, to reduce the PPG sensor size, the PPG readout electronics is embedded into the array of photo diodes PDA.

Incorporated U.S. Pat. Pub. No. US 2023/0008487 entitled Sensing System and Method for Smart Rings Employing Sensor Spatial Diversity is incorporated herein in its entirety. This additionally describes methods of operation of the ring system 100 including solutions for processing reflective and transmissive PPG sensing operations.

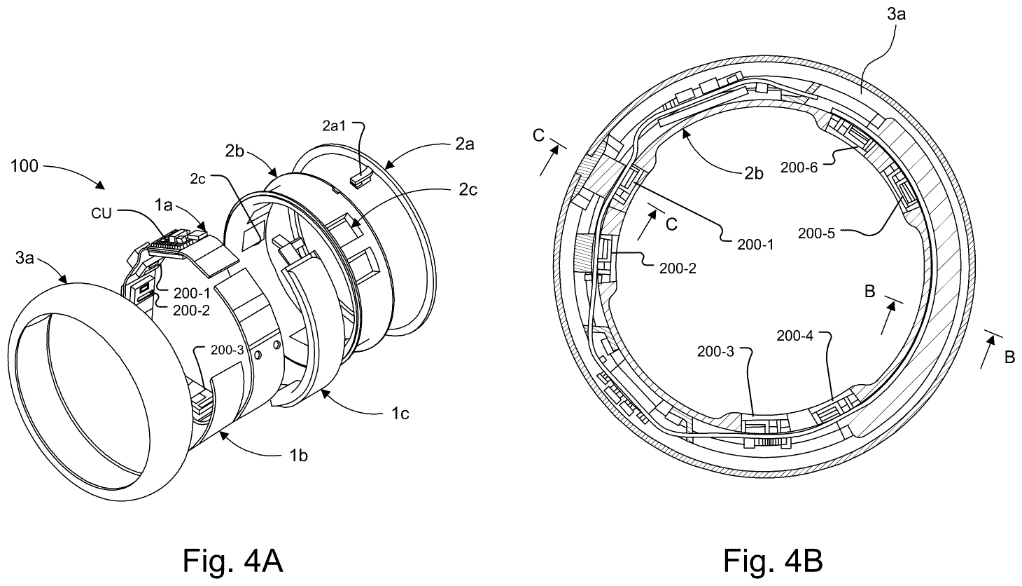

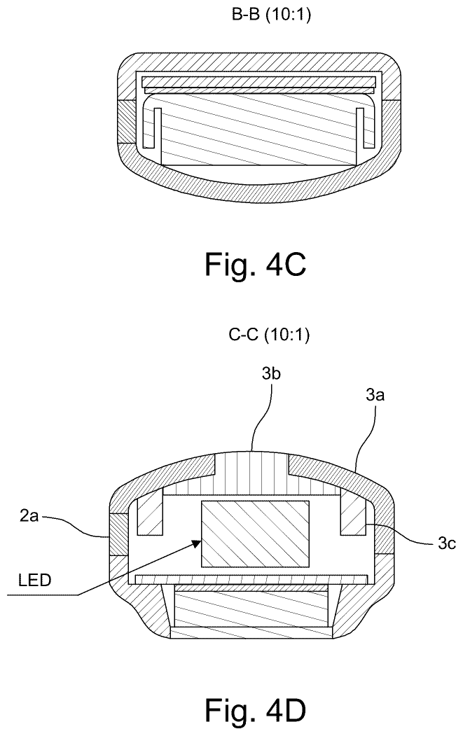

FIGS. 4A-4D is an assembly drawing showing one design for a compact smart ring.

The ring 100 includes the printed circuit board (PCB) la, ferrite inductive charging antenna 1b, battery 1c, a metal or plastic rim 2a, an inner ring 2b, windows or ports 2c in the inner ring, and an outer preferably metal shell 3a. A series of the PPG sensor units 200-1 to 200-n are integrated into the PCB 1a.

Inner ring 2b has a U shape cross-section, and is not symmetrical. It has ports or windows for optical sensors. In other examples, the profile of inner ring 2b is J, L or I shaped.

The preferred material for rim 2a is metal, but can another electrically and/or thermally conductive material and is even plastic in some examples. The rim 2a confines the printed circuit board on the inner ring 2b.

Preferably, rim 2a is first assembled onto inner ring 2b either with over molding or by gluing or other bonding technique.

Rim 2a further comprises one or more “clips” 2a1. The clips 2al of rim 2a are to be connect with and retain the PCB 1a. The outer metal parts of the outer metal shell 3a and rim 2a are used as antennas for Bluetooth-low-energy communications block BLE of the control unit CU. In one example, the outer metal shell 3a and rim 2a are connected to each other and to the PCB 1a with conductive glue.

The assembly process is preferably as follows. Rim 2a is connected to inner ring 2b either via overmolding or gluing. Then, the PCB 1a is placed on the inner ring 2b in a way that the optical sensors 200 on the PCB 1a are inserted and glued into the ports or transparent windows 2c of inner ring 2b. Then, some specific electrical contact points on the PCB are connected to rim 2a using either the metal tabs 2al on rim 2a either using conductive foam or conductive glue. Finally, the assembled group: (PCB 1a with control unit CU, ferrite charging antenna 1b, and battery 1c) along with (rim 2a, inner ring 2b, and windows 2c) are inserted into the outer metal shell 3a.

Concerning establishing an electrical contact from PCB to outer ring, the metal rim 2a is assembled to the inner ring 2b, the metal rim has a connecting means for making an electrical contact to a PCB contact. The metal rim will be a part of outer surface and part of outer ring.

The ring can optionally further include: LED light diffuser 3b, possibly the same color as outer ring 3a. LED gasket 3c can also be included. This can be a tape or similar for optical isolation of the LED to PCB and inner ring.

As shown in FIG. 4B, several optical sensors 200 are distributed around the inner circumference of the ring. Typically, more than two optical sensors are provided. The illustrated example has 6.

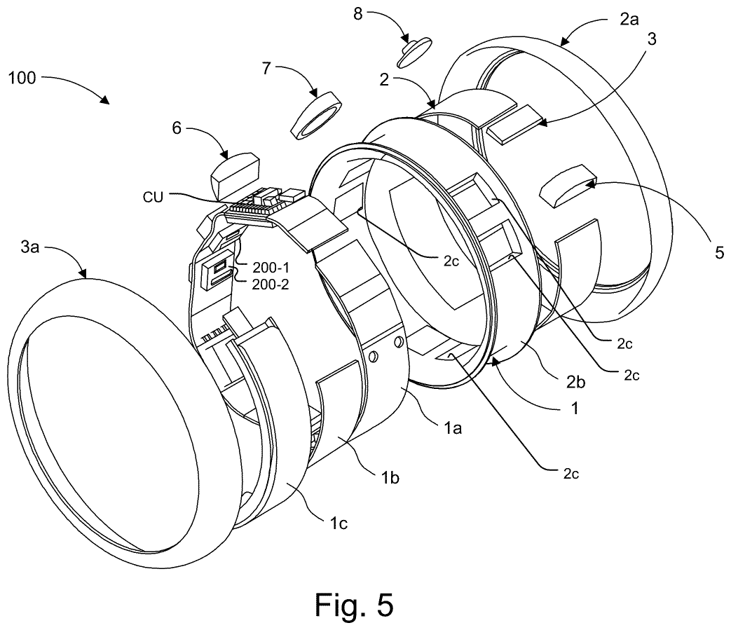

FIG. 5 is an assembly drawing showing another design for a compact smart ring.

Also shown are a series of gaskets for controlling spacing between parts and ensure waterproofing.

Reference numerals 2, 3, 5 and 6 gaskets and reference numerals 7 and 8 are insulating, light blocking gaskets.

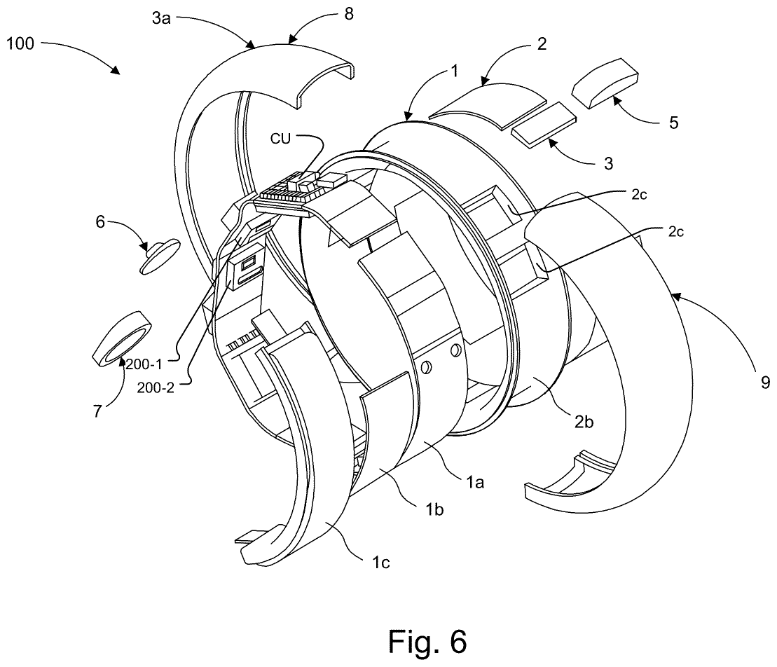

FIG. 6 is an assembly drawing showing another design for a compact smart ring.

This design employs a split outer ring. The outer ring is formed of a first part 9 and second part 10. The rim 2a is not shown.

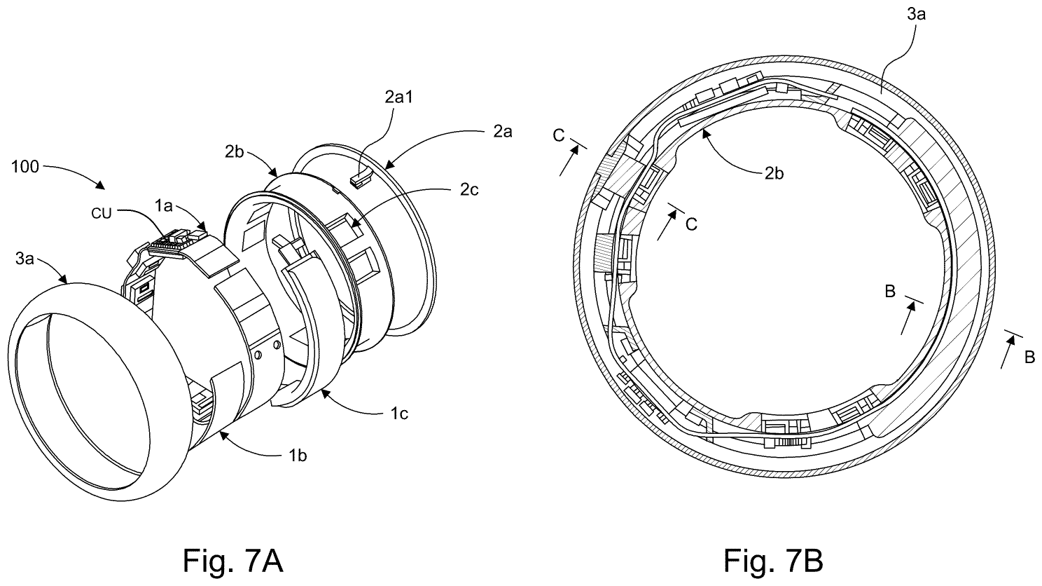

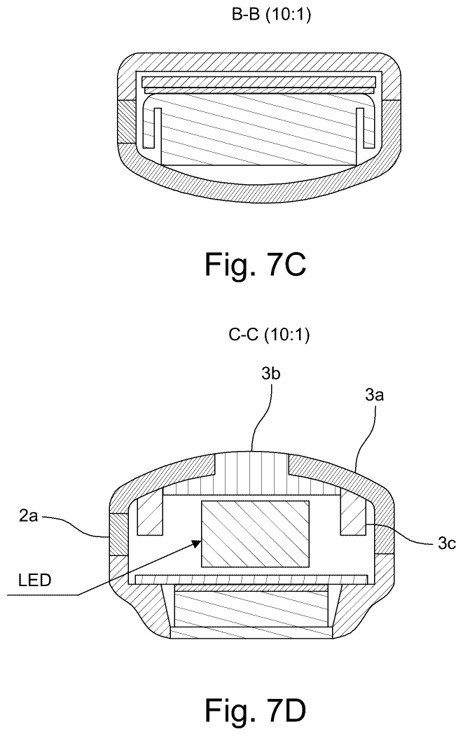

FIG. 7 is an assembly drawing showing another design for a compact smart ring.

This design employs an outer ring 3a of two half circles 8 and 9. The rim 2a is not shown.

One problem that arises is how to make an electrical or thermal contact from PCB la to outer ring 3a in a compact wearable ring structure.

It is often preferred that the structure is rigid keeping its format and size. A wearable is often having a wireless connection to a smart phone or laptop or directly to a web. Typically, the connection is Bluetooth, LoRa, Wifi or other widely used low-power radio communication solution which needs a receiver and transmitter. There is also a need for an antenna structure which is be placed on or electrically connected with the PCB 1a inside the ring structure. The outer ring structure could decrease the antenna efficiency. Providing an antenna element partly or wholly on the outer ring close or on the surface of the outer ring structure improves radio communication efficiency and thus also reduces the power consumption needed for communication. The antenna structure arranged on or encompassing the outer ring creates a requirement for electrical connection from the BLE block of the control unit CU of the PCB 1a to the outer ring 3a and/or rim 2a which then functions as a transmit and receive antenna for the data link provided by the BLE module of the control unit CU.

In other examples, both the rim 2a and shell 3a are fully plastic. In this case, the data link is established via a surface mount technology (SMT) chip antenna on the PCB 1a. This means that the antenna is an IC which is soldered on PCB like the other SMT component.

The electrical connection to the outer ring 3a can be also used for other purposes together with the antenna structure or separately. An electrode structure for enabling measuring ECG or bioimpedance can be arranged by having one electrode having on the inner surface of the ring and another electrode connection arranged on the top of the outer ring. The outer ring electrode can be touched by another hand or another body part to form two electrode measurement arrangement.

The outer ring 3a is also a key element when measuring an outside temperature using a temperature sensor set inside a ring typically placed on the PCB. It is known that internal temperature will stabilize to the temperature between skin temperature and outer ambient temperature. The time constant is however very long. Having a better thermal contact from the PCB to the outer surface of the ring the temperature measurement can ensure faster and more accurate readings.

This is solved with an outer rim 2a that is designed to make a connection from the PCB 1a and/or inner ring 2b to the outer ring 3a of the ring. The outer rim 3a is electrical and/thermally conductive material such as metal or plastic or metalized plastic. The rim 3a can be made of metal such as aluminum, titanium, copper, stainless steel, gold, silver or composite. The outer rim 3a may have connect features for making an electrical and/or thermal contact to a PCB contact on PCB 1a. The outer rim 3a will be a part of outer surface and part of outer ring. The connecting of the outer rim 3a to the inner ring 2b can be made by gluing using a clip, soldered or welded or having such a tight physical pressing contact.

The material for the outer rim 3a and connecting element and attaching material can be selected so that the outer rim is well connected electrically and thermally (using for example, stainless steel, silver, gold, copper, etc.) or it is conducting electrically but not thermally (using for example thermally isolating but conductive materials such as ceramics or ceramic composite, or it is connected thermally only (using for example thermal plastics such as polyimide or other TCEI material).

The outer rim 3a may have a tab or clip or other connecting element or multiple such elements. These elements can be a fixed part of the outer rim 3a, or they have been glued, soldered otherwise attached to the outer rim. The outer rim 3a is then fixed to the inner ring 2b and/or to the PCB 1a by soldering or gluing using a conductive glue or using a conductive foam, having a mechanically tight connecting such pressing or spring type connection.

FIGS. 7A-7D illustrate the another design for a compact smart ring.

Here, the PCB 1a with electrical components is flexible. The ferrite charging antenna 1b is mounted to an outer side of PCB 1a. The battery 1c is also flexible and mounted to an outer side of the PCB 1a. The outer rim 2a provides electrical/thermal connections to the inner ring 2b and/or the PCB 1a, with a resistance of preferably less than 100 milliohm. It is possible that the outer rim 2a comprises one or more tabs 2al (as shown on the upper part of the rim) to make connection to the PCB 1a. These may be soldered or glued or use a conductive foam. Inner ring 2b has windows 2c for providing optical pathway from the LED/optical receiver on the PCB to the skin/finger of the user. The windows 2c can be openings, covered by an epoxy or similar coating, or over molded into the inner ring with a transparent or translucent material. Outer ring 3a covers the assembly. The ring can optionally further include: LED light diffuser 3b, possibly the same color as outer ring 3a. LED gasket 3c can also be included. This can be a tape or similar for optical isolation of the LED to PCB and inner ring.

Generalizing aspects can feature a ring wearable device having a connection from the outer surface of the ring to the inner ring structure comprising: an outer rim 2a having a connecting means for making an electrical and/or thermal contact to PCB or inner ring; the outer rim is connected with the connections to the PCB. Further, the outer rim will be a part of the outer surface part of the outer ring.

Aspects also related to a method of proving a connection from the outer surface of the ring to the inner ring structure comprising in a ring wearable device.

Other aspects include:
The outer rim 2b is fabricated from electrical and/thermally conductive material such as metal or plastic.
The outer rim 2b can be made of metal such as aluminum, titanium, copper, stainless steel, gold, silver or composite.
The outer rim 2b has connections for making an electrical and/or thermal contact to a PCB contact in some examples.
The connecting of the outer rim to the outer ring can be made by gluing using a clip, soldered or welded or having such a tight physical pressing contact.
The material for the outer rim and connecting element and attaching material can be selected so that the outer rim: is connected electrically and thermally (using for example, stainless steel, silver, gold, copper, etc.) or is conducting electrically but not thermally (using for example thermally isolating but conductive materials such as ceramics or ceramic composite or is connected thermally only (using for example thermal plastics such as polyimide or other TCEI material).

The outer rim 2a may have a tab or clip or other connecting element or multiple such elements 2a1. These elements can be a fixed part of the outer rim, or they have been glued, soldered otherwise attached to the outer rim.

The element can be fixed to the inner ring or to the PCB by soldering or gluing using a conductive glue or using a conductive foam, having a mechanically tight connecting such pressing or spring type connection.

The metal rim will often be part of the outer ring, and it may be connected electrically and or thermally to the other outer ring.

The electrical contact is designed for using the rim or the outer ring as an antenna, or measuring electrode.

Thermal contact is designed to make a good thermal contact from the surface to the temperature sensor on the PCB.

The outer rim can be a full ring, part ring and sector of the ring as described previously.

One technical problem that arises is how to assembly a ring structure comprising an inner ring 2b, PCB layer 1a and outer ring 3a, the PCB including electronic components (possible preformed to a circular form).

There are currently numerous wearable devices designed for measuring and monitoring user's health or activity or environment around the user. Different form factors are used such as a wrist device, armband or ring used on user's finger. A ring form provides many practical advantages for use. However, it is also challenging structure with small dimensions without extending parts or elements and surface being smooth and flat over connected parts. It is often preferred that the structure is rigid keeping its format and size. A wearable is often having a wireless connection to a smart phone or laptop or directly to a web.

The ring construction is challenging from an assembly standpoint. It is small and typically has a rigid form. Further, due to being in a direct skin contact, it must be closed properly and to have a smooth surface. When the electronics are placed, the ring structure's use of space is very critical. Also, the PCB and components are needed to be set carefully and in right place.

The solution involves a method to assembly and a device. First, an inner ring has a groove which is not higher than (has no height over) the components on the PCB (the groove is not enclosing PCB and components). The PCB is then set to the groove. Third, a (metal) rim is assembled to the inner ring so that at least one side of the groove is over the height of the PCB and its components. Fourth, the outer ring is assembled to the inner ring and the rim to form a full housing to the PCB and its components.

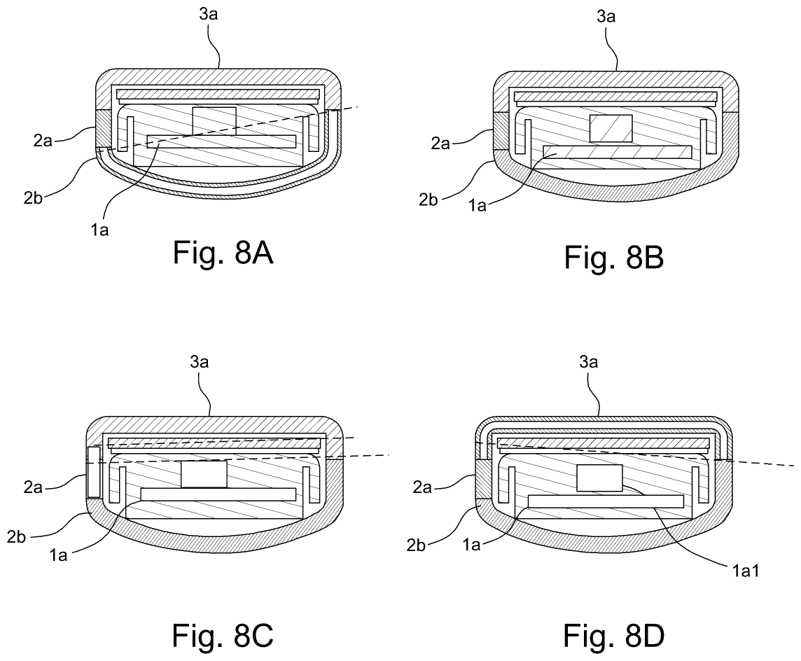

FIGS. 8A, 8B, 8C, and 8D show four potential cross sections of the ring 100. Here, the inner ring 2b, PCB 1a, other electrical components such as the battery and ferrite, rim 2a, and outer ring 3a are shown. The outer ring 3a may have groove in some examples.

Accordingly, in some aspects, a method for manufacturing a ring wearable device comprises several steps. First, an inner ring 2b has a groove which is not higher than (has no height over) the components on the PCB 1a (the groove does not enclose the PCB and associated components). Second the PCB 1a is set to the groove. Third, a (metal) rim 2a is assembled to the inner ring so that at least one side of the groove is over the height of the PCB and its components. Fourth, the outer ring 3a is assembled to the inner ring 2b and the rim 2a to form a full housing to the PCB and its components.

Different aspects can have different characteristics. The inner ring 2b may have a profile like “J”, “L” or “I”. The material of the inner or outer ring can be ceramic, plastic or metal. The inner ring can be made of multiple ring parts or partial rings. The rim can be ceramic, plastic or metal. The outer ring or rim may have connections for making an electrical and/or thermal contact to a PCB contact. The connection of the rim 2a to the outer ring 3a can be made by gluing using a clip, soldered or welded or having such a tight physical pressing contact. The rim 2a be fixed to the inner ring 2b by soldering or gluing using a non-conductive or conductive glue or using a non-conductive conductive foam, or having a mechanically tight connecting such pressing or spring type connection. The outer ring 3a can be formed from two or more ring parts. The outer ring 3a can also be formed of two or more sectional ring parts.

Accordingly, in some aspects, another method for manufacturing a ring wearable device 100 comprises several steps. First, an inner ring 2b has a groove which is not higher than (has no height over) the components on the PCB 1a (the groove is not enclosing PCB and components). Second the PCB 1a is set to that groove. Third, a (metal) rim 2a is assembled to the inner ring 2b so that at least one side of the groove is over the height of the PCB 1a and its components. Fourth, the outer ring 3a is assembled to the inner ring and the rim forms a full housing for the PCB 1a and its components.

Different aspects can have different characteristics. The inner ring 2b again may have the profile like “J”, “L” or “I”. The material of the inner ring 2b and/or outer ring 3a can be ceramic, plastic or metal. The inner ring 2b can be made of multiple ring parts or partial rings. The rim 2a can be ceramic, plastic or metal. The outer ring 3a or rim 2a may have connectors for making an electrical and/or thermal contact to a contact of the PCB 1a. The connection of the rim 2a to the outer ring 3a can be made by gluing using a clip, soldered or welded or having such a tight physical pressing contact. The rim 2a can be fixed to the inner ring 2b by soldering or gluing using a non-conductive or conductive glue or using a non-conductive or conductive foam, or having a mechanically tight connecting such pressing or spring type connection. The outer ring 3a can be formed of two or more ring parts. The outer ring can be formed of two or more sectional ring parts.

Aspects also extend to methods of assembly a ring structure comprising an inner ring, PCB layer and outer ring, the PCB including electronic components (possible preformed to a circular form).

There are currently numerous wearable devices designed for measuring and monitoring user's health or activity or environment around the user. Different form factors are used such as a wrist device, armband or ring used on user's finger. A ring form provides many practical advantages for use. However, it is also challenging structure with small dimensions without extending parts or elements and surface being smooth and flat over connected parts. It is often preferred that the structure is rigid keeping its format and size. A wearable is often having a wireless connection to a smart phone or laptop or directly to a web.

The ring construction is challenging to assembly. It is small and typically has a rigid form. Further, due to being in direct skin contact, it must be closed properly and to have a smooth surface. When the electronics are placed inner the ring structure the use of space is very critical. Also, PCB and components are needed to be set carefully and in right place.

According to one approach, first, an inner ring has a non-symmetric groove, one side having the wall height higher that the height of the components on the PCB, and another side having the wall height lower than the height of the components on the PCB (i.e. the groove is not enclosing PCB and components). The PCB is set to the groove. Third, an outer ring is assembled to the inner ring to form a full housing to the PCB and its components.

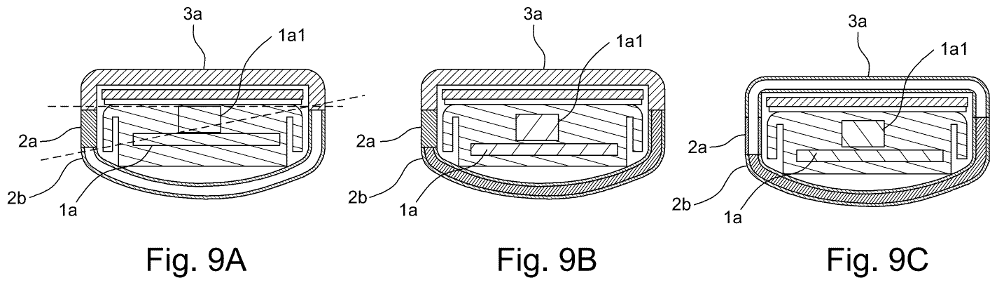

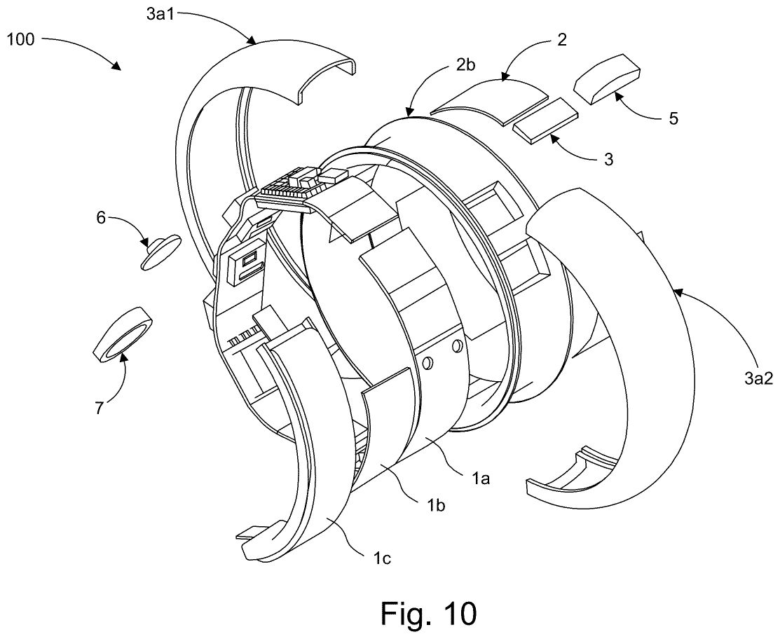

FIGS. 9a, 9B, and 9C shows three potential cross sections of the ring. In the device, the structure is shown including inner ring 2b, PCB 1a, PCB electrical component 1a1, including a battery, a ferrite, and outer ring 3a.

Aspects also extend to another method for manufacturing a ring wearable device including several steps. First, an inner ring 2b has a non-symmetric groove, of which one side has a wall height higher that the height of the components 1a1 on the PCB 1a, and another side having the wall height lower than the height of the components 1a1 on the PCB 1a (i.e. the groove is not enclosing PCB and components). Second the PCB 1a is set to the groove. Third, the outer ring 3a is assembled to the inner ring 2b and the rim 2a to form a full housing to the PCB and its components.

There can be further aspects. The inner ring may have a profile like “J”, “L” or “I”. The material of the inner or outer ring can be ceramic, plastic or metal. The inner ring can be made of multiple ring parts or partial rings. The outer ring may have connecting means for making an electrical and/or thermal contact to a PCB contact. The outer ring can be formed of from two or more ring parts. The outer ring can be formed of two or more sectional ring parts.

Additional aspect also related to an assembly method for electronic ring device, and a ring device arrangement. This addresses technical problem of how to assembly a ring structure comprising an inner ring, PCB layer and outer ring, the PCB including electronic components (possible preformed to a circular form).

The solution includes several steps. First, an inner ring has a non-symmetric or symmetric groove. The PCB and electrical components are set to the groove. Third, an outer ring is assembled to the inner ring with at least two sectional parts, the sectional parts forming a full outer ring.

FIG. 10 is an assembly drawing showing another design for a compact smart ring. This drawing shows inner ring 2b, PCB 1a, and a two part outer ring 3al and 3a2.

According to other aspects, another method for manufacturing a ring wearable device comprises several steps. First, an inner ring 2b has a non-symmetric or symmetric groove. Second, the PCB 1a is set to the groove. Third, an outer ring 3al and 3a2 is assembled over the inner ring 2b, the outer ring comprising at least two sectional parts 3al and 3a2, the sectional parts forming a full outer ring.

The ring can further have additional aspects. The inner ring 2b may have the profile like “U”, “J”, “L” or “I”. The material of the inner or outer ring can be ceramic, plastic or metal. The inner ring can be made of multiple ring parts or partial rings. The outer ring 3a may have connector for making an electrical and/or thermal contact to a contact of PCB 1a. The outer ring can be formed of two or more ring parts as shown. The outer ring 3a comprises two half circle sectional parts. The outer parts are connected to the inner part using gluing, screws or snap-on elements. The outer parts are connected to each other by gluing, screws or snap-on elements. The PCB may run on the inner ring area of the two outer ring parts (but PCB may not have components on it).

While this invention has been particularly shown and described with references to preferred embodiments thereof, it will be understood by those skilled in the art that various changes in form and details may be made therein without departing from the scope of the invention encompassed by the appended claims.

What is claimed is:

1. A ring system, comprising:
a printed circuit board;
an inner ring holding the printed circuit board; and
an outer shell mounted over the printed circuit board and the inner ring.

2. The ring system as claimed in claim 1, further comprising ports in the inner ring providing optical paths for optical sensors of the printed circuit board.

3. The ring system as claimed in claim 1, wherein the inner ring has a U shape cross-section.

4. The ring system as claimed in claim 1, further comprising a rim assembled onto the inner ring for confining the printed circuit board on the inner ring.

5. The ring system as claimed in claim 1, wherein the outer shell is metal.

6. The ring system as claimed in claim 1, wherein the outer shell is metal is electrically connected to the printed circuit board to function as an antenna.

7. The ring system as claimed in claim 1, further comprising at least two and preferably six or more optical sensors of the printed circuit board distributed around an inner circumference.

8. The ring system as claimed in claim 1, wherein the outer shell is provided in two pieces.

9. An assembly process for a ring, comprising:
laying a PCB on an inner ring;
aligning optical sensors of the PCB with ports of the inner ring; and
inserting an assembly including the inner ring and PCB into an outer shell.

10. The process as claimed in claim 9, further comprising connecting a rim to the inner ring via a bonding process.

11. The process as claimed in claim 9, further comprising providing electrical connections between the PCB and the outer shell to function as an antenna.

12. The process as claimed in claim 9, further comprising employing clamps to retain the PCB in the inner ring.

13. The process as claimed in claim 9, further comprising electrically connecting the outer shell and the rim with conductive glue.

14. The process as claimed in claim 9, wherein the outer shell is provided in two pieces.
